(19) United States
(12) Patent Application Publication
(10) Pub. No.: US 2004/0076952 A1
(43) Pub. Date: Apr. 22, 2004

(54) Method and apparatus for continuous amplification of DNA

(76) Inventor: Toshio Kawai, Kawanishi-shi (JP)

Correspondence Address: JUDGE PATENT FIRM, RIVIERE SHUKUGAWA 3RD FL., 3-1 WAKAMATSU-CHO, NISHINOMIYA-SHI, HYOGO, 662-0035, JP

(21) Appl. No.: 10/065,456

(22) Filed: Oct. 19, 2002

(51) Int. Cl. C12Q 1/68; C12P 19/34

(52) U.S. Cl. 435/6; 435/91.2

(57) ABSTRACT

Continuous DNA-amplification method and apparatus. Reaction mixture containing reagent solution and DNA fragments serving as templates is continuously temperature-processed by heat-exchange fluids whose temperatures differ, enabling successive DNA amplification reactions to be carried out efficaciously in large volume. Apparatus has a reaction-mixture tank, isothermal denaturing, annealing and elongation tanks, a recirculation path that circuits the isothermal tanks, and a pump that works to feed reaction mixture within the recirculation path unidirectionally through it. The reaction mixture, transferred from tank to tank by the pump, is maintained at prescribed reaction temperatures in the isothermal tanks. Heat-exchange efficiency is improved compared with batch systems, and the reaction mixture—uniformly, swiftly put into prescribed temperature states—can be amplification-reacted under ideal conditions, enabling quantum improvement in the amplification efficiency. Amplification reactions are carried out by circuit-feeding the reaction mixture, which serves to establish per-cycle DNA amplification quantity at large volumes.

### BACKGROUND OF INVENTION

1. Technical Field

The present invention relates to DNA amplification using the polymerase-chain reaction (PCR) method; in particular the invention relates to a method and apparatus for continuous PCR-based amplification of DNA.

2. Description of the Related Art

DNA amplification techniques based on the PCR method are for the most part carried out using a palette into which a set of tubes is loaded. The DNA is amplified by heating the palette and tubes with a heater, or blowing air of a prescribed temperature on them; maintaining, for prescribed lengths of time, the temperature of the reaction solution inside the tubes at a denaturation temperature, an annealing temperature, and an elongation (extension) temperature; and then repeating this reaction cycle. Apparatuses of this kind have been made known to the public—for example, in Japanese Pat. App. Pub. No. H9-262084.

With the PCR method, DNA is amplified by maintaining the reaction solution at the given temperatures and repeatedly carrying out the denaturing, annealing, and extension reactions; but heat exchange with the reaction solution cannot be carried out efficaciously by the conventional amplification techniques, because the temperature is adjusted by heating the palette and tubes with a heater, or by blowing air of a given temperature on them. Moreover, getting the tubes that are loaded into the palette to be one by one under uniform temperature conditions is difficult. The consequent problem has been that without being able to react all of the reaction solution under ideal conditions, the amplification efficiency cannot be improved. Likewise, because DNA amplification is carried out in batch form, the amount of DNA that may be amplified at one time is limited, which unavoidably puts the costs required for amplification higher.

### SUMMARY OF INVENTION

An object of the present invention is a method as well as an apparatus for continuous amplification of DNA that feeds a reaction solution along a recirculation path, and that along the way heat-exchanges, inside isothermal tanks, the reaction solution within the recirculation path so that it can be maintained at prescribed temperatures, enabling efficient, large-volume amplification of DNA.

A method for continuous amplification of DNA set out by the present invention feeds, with a recirculation path and a pump furnished in the recirculation path, unidirectionally through it a reaction mixture containing DNA fragments and a reagent solution, held in a reaction-mixture tank. The reaction mixture within the recirculation path is sent by way of, in the following recited order: a denaturing isothermal tank in which a temperature for dissolving apart the DNA's double strands is maintained; an annealing isothermal tank in which a temperature at which primers contained in the reagent solution anneal to the DNA fragments is maintained; and an elongation isothermal tank in which a temperature at which complementary chains are extended continuously onto the primers is maintained. The method is then characterized in that the reaction mixture is maintained, with heat-exchange fluids within the isothermal tanks, for prescribed times at prescribed temperatures, and is then recirculated into the reaction-mixture tank, to repeat the amplification reactions continuously.

The foregoing continuous amplification method sets out performing the amplification reactions by taking the time for heat-exchange in the denaturing isothermal tank as a reference time, and setting the individual heat-exchange times in the annealing isothermal tank and the elongation isothermal tank as multiples of the reference time.

A separate method for continuous amplification of DNA set out by the present invention circuit-feeds unidirectionally through an endless recirculation path, using a pump furnished therein, a reaction mixture containing DNA fragments and a reagent solution, held within the recirculation path. The reaction mixture within the recirculation path is circulated by way of, in the following recited order: a denaturing isothermal tank in which a temperature for dissolving apart the DNA's double strands is maintained; an annealing isothermal tank in which a temperature at which primers contained in the reagent solution anneal to the DNA fragments is maintained; and an elongation isothermal tank in which a temperature at which complementary chains are extended continuously onto the primers is maintained. The method is then characterized in that the reaction mixture is maintained, with heat-exchange fluids within the isothermal tanks, for prescribed times at prescribed temperatures to repeat the amplification reactions continuously.

An apparatus for continuous amplification of DNA set out by the present invention is equipped with: a reaction-mixture tank for holding a reaction mixture containing DNA fragments and a reagent solution; a denaturing isothermal tank for holding a heat-exchange fluid adjusted to a temperature for dissolving apart the DNA's double strands; an annealing isothermal tank for holding a heat-exchange fluid adjusted to a temperature at which primers contained in the reagent solution anneal to the DNA fragments; an elongation isothermal tank for holding a heat-exchange fluid adjusted to a temperature at which complementary chains are extended continuously onto the primers; a recirculation path through which the reaction mixture in the reaction-mixture tank is fed/guided; and a pump that works to feed the reaction mixture in the recirculation path unidirectionally through it. The recirculation path is arranged such that it circuits from the reaction-mixture tank and goes by way of the denaturing isothermal tank, the annealing isothermal tank, and the elongation isothermal tank back to the reaction-mixture tank. The apparatus is thus configured such that the reaction mixture in the recirculation path is for timed intervals maintained at prescribed temperatures determined by the heat-exchange fluids in the isothermal tanks.

The above-noted denaturing isothermal tank, annealing isothermal tank, and elongation isothermal tank respectively include: container bodies that hold the heat-exchange fluids; heat sources that heat the heat-exchange fluids to, and retain them at, prescribed temperatures; and stirring devices that stir the heat-exchange fluids.

It can be that the denaturing isothermal tank, annealing isothermal tank, and elongation isothermal tank respectively include: container bodies that hold the heat-exchange fluids; stirring devices that stir the heat-exchange fluids; and heating devices that supply the heat-exchange fluids to the container bodies; with the heating devices each containing a pump that circuit-feeds the heat-exchange fluids in between the container bodies and the heating devices, and heat sources that heat the heat-exchange fluids to, and retains them at, prescribed temperatures.

A plurality of recirculation paths in parallel may be provided along with the pump between the reaction-mixture tank and the container bodies.

The denaturing isothermal tank, annealing isothermal tank, and elongation isothermal tank may be provided in plural sets, and a plurality of recirculation paths in parallel may be provided along with the pump between the reaction-mixture tank and the isothermal tanks of the plural sets.

Coiled heat-exchange paths immersed into the each of the isothermal tanks may be provided in sections along the way of the recirculation path(s).

From the following detailed description in conjunction with the accompanying drawings, the foregoing and other objects, features, aspects and advantages of the present invention will become readily apparent to those skilled in the art.

### BRIEF DESCRIPTION OF DRAWINGS

FIG. 1 is an outline sectional view illustrating principles of a continuous amplification apparatus;

FIG. 2 is a sectional view illustrating an isothermal tank and a heating device in a separate embodiment, with the recirculation path shown fragmentarily;

FIG. 3 is a sectional view illustrating a separate embodiment in which the mode of circulating the reaction mixture has been altered, wherein the recirculation path is shown fragmentarily; and

FIG. 4 is a plan view of a continuous amplification apparatus, illustrating recirculation paths in a modified example thereof.

### DETAILED DESCRIPTION

By the method for continuous amplification of DNA under the present invention, the reaction mixture within the recirculation path is transfer-maneuvered by the pump, and by heat-exchange carried out in the denaturing isothermal tank, annealing isothermal tank, and elongation isothermal tank, each adjusted to respectively different temperatures, the reaction mixture is maintained in prescribed temperature states. The method improves the heat-exchange efficiency compared with conventional amplification methods by which a palette and tubes have been heated with a heater, or the temperature adjusted by blowing air of a prescribed temperature on them. The method moreover can suppress lag in the time for the reaction mixture to go from temperature to temperature, and can uniformly and strictly maintain the reaction mixture within the recirculation path in prescribed temperature states. Reacting all of the reaction mixture under ideal conditions consequently enables the amplification efficiency to be improved by a great stride. Likewise, inasmuch as the amplification reactions are carried out circuit-feeding the reaction mixture, the DNA amplification amount per cycle may be freely set. DNA can consequently be amplified to large volume and yet simply, and the cost required for amplification can be remarkably curtailed, compared with the conventional batch systems.

Making it so that the amplification reactions are performed by making the time for heat-exchange in the denaturing isothermal tank a reference time, and setting the individual heat-exchange times in the annealing isothermal tank and the elongation isothermal tank as multiples of the reference time enables accurately controlling the heat-exchange time in the isothermal tanks merely by furnishing the pump in any of the recirculation paths. Furnishing each of the respective isothermal tanks with a buffer tank and feed pump is therefore unnecessary, and insofar as this simplifies the amplification-device structure overall, the cost of introducing the amplification device can be curtailed.

In the separate method for continuous amplification of DNA set out by the present invention, continuously amplification reactions are repeated by using the pump to circuit-feed in a single direction the reaction mixture held within the endless recirculation path; by circulating it by way of, in order: a denaturing isothermal tank, an annealing isothermal tank, and an elongation isothermal tank; and by maintaining it, with heat-exchange fluids within the isothermal tanks, for prescribed times at prescribed temperatures. The reaction mixture transferred from the elongation isothermal tank is therefore directly recirculated to the denaturing isothermal tank, enabling the reaction mixture with which the recirculation paths are replete to be repeatedly and efficaciously amplified, and improving the amplification efficiency.

Container bodies that hold the heat-exchange fluids, heat sources that heat the heat-exchange fluids to, and retain them at, prescribed temperatures, and stirring devices0 that stir the heat-exchange fluids being incorporated respectively into the denaturing isothermal tank, annealing isothermal tank, and elongation isothermal tank serves to suppress local temperature irregularities in the heat-exchange fluids within the isothermal tanks. This allows heat exchange between the reaction mixture and the heat-exchange fluids to take place under uniform temperature conditions, and enables reacting all of the reaction mixture under ideal conditions to improve the amplification efficiency.

By means of a continuous amplification apparatus furnished with heating devices separately from the container bodies that hold the heat-exchange fluids, and made so that heat-exchange fluids adjusted to prescribed temperatures by the heating devices are fed to the container bodies, the temperature state of the heat-exchange fluids within the container bodies can with exactness be equalized. Effective DNA amplification can be carried out insofar as heat exchange between the reaction mixture and the heat-exchange fluids within the heat-exchange paths consequently can take place uniformly and moreover efficaciously. The fact that the temperature of the heat-exchange fluids is adjusted on the heating-devices end serves to solve the problem of heat-exchange fluids in the vicinity of the heat sources becoming inordinately high in temperature—unavoidable wherein the isothermal tanks are each provided with a heat source—and serves to nullify problems such as the activity of the polymerase contained in the reaction mixture located in the vicinity of the heat sources being compromised.

Inasmuch as furnishing in between the reaction-mixture tank and each of the container bodies a plurality of parallel recirculation paths along with the pump means that DNA amplification can be carried out by feeding the reaction mixture to each of the recirculation paths, replicate DNA can be amplified very productively; and if necessary, amplification of different DNA fragments can be carried out simultaneously in each of the systems.

Providing the denaturing isothermal tank, annealing isothermal tank, and elongation isothermal tank in plural sets, and furnishing in between the reaction-mixture tank and the isothermal tanks of each of the plural sets a plurality of parallel recirculation paths along with the pump enables different temperature states and different heat-exchange times to be established in each of the recirculation paths, and enables simultaneously reacting for amplification a plurality of types of reaction mixture under different temperature conditions.

Providing coiled heat-exchange paths in sections along the way of the recirculation path(s) and immersing the heat-exchange paths into the each of the isothermal tanks to carry out heat exchange between the reaction mixture and the heat-exchange fluids serves to increase, by the coil length of the heat-exchange paths, the amount of reaction mixture that can be heat-exchanged in the isothermal tanks. This consequently enables simultaneously reacting larger volumes of reaction mixture, to carry out DNA amplification very efficiently.

Embodiments

Embodiment 1

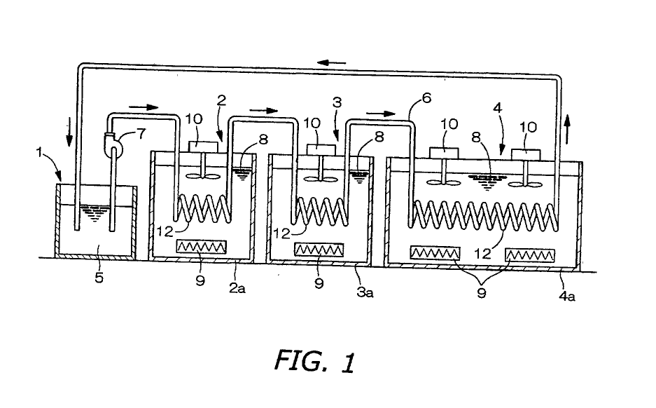

FIG. 1 represents an embodied example of an apparatus in terms of the present invention for continuous amplification of DNA. The continuous amplification apparatus in FIG. 1 includes a reaction-mixture tank 1, a denaturing isothermal tank 2, an annealing isothermal tank 3, an elongation isothermal tank 4, and comprises a recirculation path 6 arranged to circuit these tanks 1, 2, 3 and 4, and through which the reaction mixture 5 in the reaction-mixture tank 1 is fed/guided, and a pump 7 that intermittently feeds the reaction mixture 5 within the recirculation path 6 unidirectionally through it. DNA fragments that serve as templates, and a reaction mixture 5 containing a reagent solution, are held in the reaction-mixture tank 1. The reaction solution is prepared by mixing, for example, DNA primers that are made synthetically, the four kinds of dNTP bases, polymerase that is a heat-resistant enzyme, purified water, and a pH-adjusting buffer.

A heat-exchange fluid 8 is held in each one of the denaturing, annealing, and elongation isothermal tanks 2, 3 and 4. The isothermal tanks 2, 3 and 4 are individually composed of: respective container bodies 2a, 3a and 4a that hold the heat-exchange fluid 8; sheath heaters (heat sources) 9 that heat the heat-exchange fluid 8 to, and retain it at, prescribed temperatures; stirring devices 1 0 that stir the heat-exchange fluid 8; and, out of the figure, temperature sensors and control circuitry that on/off controls the sheath heaters 9 based on output signals from the temperature sensors. The heat-exchange fluid 8 in the denaturing isothermal tank 2 is adjusted to a temperature (95° C.) for dissolving apart the DNA's double strands. The heat-exchange fluid 8 in the annealing tank 3 is adjusted to a temperature (50° C.) at which the primers contained in the reagent solution anneal to the DNA fragments. The heat-exchange fluid 8 in the elongation isothermal tank 4 is adjusted to a temperature (72° C.) at which complementary chains are extended continuously onto the primers.

The recirculation path 6 is formed of thin-walled tubing made of a plastic such as polytetrafluoroethylene having heat-resistant properties, or tubing of a metal such as copper, or especially stainless steel, whose thermal conductivity is favorable; and sections immersed into the isothermal tanks 2, 3 and 4 are shaped into coil form as heat-exchange paths 1 2. The inlet end and the outlet end of the recirculation path 6 are each immersed into the reaction mixture 5 in the reaction-mixture tank 1; and with the pump 7 arranged in the path toward the inlet end, the reaction mixture 5 is intermittently circuit-fed from the inlet end, by way of the isothermal thanks 2, 3 and 4, and into the reaction-mixture tank 1.

A continuous amplification apparatus configured as described above repeats amplification reactions continuously by intermittently feeding with the pump 7 the reaction mixture 5 held in the reaction-mixture tank 1 unidirectionally via the recirculation path 6, passing the reaction mixture 5 within the recirculation path 6 through the denaturing isothermal tank 2, the annealing isothermal tank 3, and the elongation isothermal tank 4, in that order, and after maintaining it for prescribed times at prescribed temperatures in the isothermal tanks 2, 3 and 4, recirculating it into the reaction-mixture tank 1. The time for heat exchange between the reaction mixture 5 and the heat-exchange fluid 8 in the denaturing isothermal tank 2 is put at approximately 30 seconds. The time for heat exchange between the reaction mixture 5 and the heat-exchange fluid 8 in the annealing isothermal tank 3 is put at approximately 30 seconds. The time for heat exchange between the reaction mixture 5 and the heat-exchange fluid 8 in the elongation isothermal tank 4 is put at approximately 60 to 120 seconds. These heat-exchange times may be accurately defined according to the time that the pump 7 is at rest. Because the heat-exchange times should be altered according to the compositions of the DNA fragments and the reaction mixture 5, the times are not limited to those just illustrated.

Embodiment 2

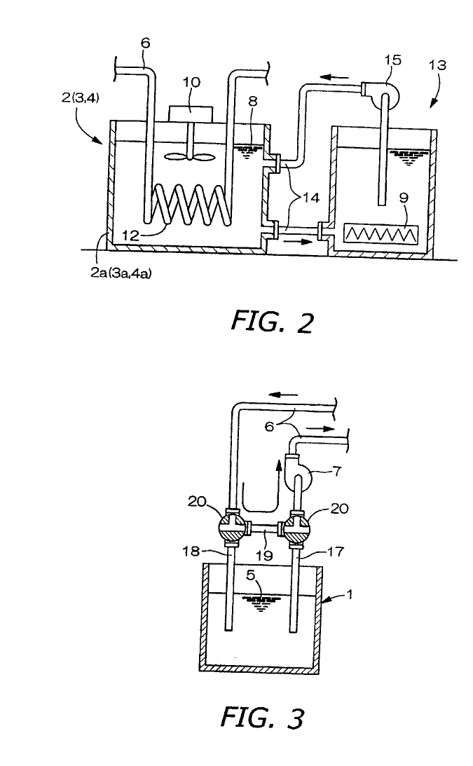

Each of the denaturing 2, annealing 3, and elongation 4 isothermal tanks may be composed of, as depicted in FIG. 2: the respective container bodies 2a, 3a and 4a that hold the heat-exchange fluid 8; stirring devices 1 0 that stir the heat-exchange fluid 8; heating devices 1 3 that supply the heat-exchange fluid 8 to the container bodies 2a, 3a and 4a; and a pair of paths 1 4 by which the container bodies 2a, 3a and 4a and heating devices 1 3 communicate. The heating devices 1 3 are composed of pumps 1 5 that via the paths 1 4 circuit-feed the heat-exchange fluid 8 between the container bodies 2a, 3a and 4a and the heating devices 1 3, and the sheath heaters (heat sources) 9 that heat the heat-exchange fluid 8 to, and retain it at, prescribed temperatures. In this way furnishing the heating devices 1 3 separately from the container bodies 2a, 3a and 4a and adjusting the temperature of the heat-exchange fluid 8 on the heating-devices 1 3 end eliminates temperature irregularities in the heat-exchange fluid 8 within the container bodies 2a, 3a and 4a, and enables uniform, efficacious heating or cooling of the reaction mixture 5 within the heat-exchange paths 12.

Embodiment 3

The continuous amplification apparatus may be embodied by partially altering Embodiment 1, as depicted in FIG. 3. In this case an inlet path 1 7 and an outlet path 1 8 are connected by a bypass path 1 9, and three-way directional control valves 20 are arranged in the respective connecting portions between the two paths 1 7, 1 8 and the bypass path 1 9. In this embodiment, after the recirculation path 6 is replete with the reaction mixture 5, the three-way directional control valves 20 are switched to render the recirculation path 6 a path in endless form, and the reaction mixture 5 is intermittently circuit-fed in a single direction with the pump 7, allowing the reaction mixture 5 to be heat-exchanged in the isothermal tanks 2, 3 and 4, making it likewise as with Embodiment 1. When the reactions have ended, the three-way directional control valve 20 on the outlet-path 1 8 end is opened to bring out the reaction mixture 5, and an iteration of the above-described reaction cycle is carried out over again.

Embodiment 4

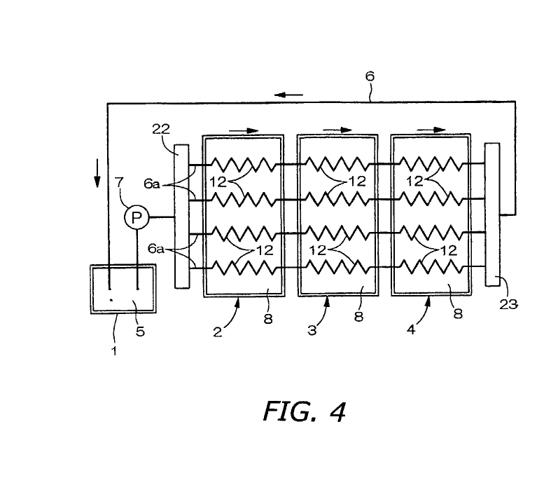

FIG. 4 represents a still different embodied example of a continuous amplification apparatus. Therein, a plurality of recirculation-path 6a systems is furnished in parallel between the reaction-mixture tank 1, and the isothermal tanks 2, 3 and 4, making the apparatus able simultaneously to process for amplification a large volume of reaction mixture S. In this case, a branching manifold 22 is arranged in between the plurality of recirculation-path 6a systems and the pump 7, and furthermore a collecting manifold 23 is arranged on the terminal end of the plurality of recirculation-path 6a systems.

Apart from the foregoing, the denaturing isothermal tank 2, annealing isothermal tank 3, and elongation isothermal tank 4 may be furnished in plural sets, and a plurality of recirculation-path 6a systems in parallel may be furnished along with the pump 7 between the reaction-mixture tank 1 and the plural sets of isothermal tanks 2, 3 and 4. The pump 7, moreover, may be constituted by a squeeze-type pump, and its inlet and outlet may respectively be connected to the recirculation path 6/recirculation-path 6a systems. The recirculation-path 6/recirculation-path 6a systems may be composed of thin-film tubing and a metal layer laminated on to at least the external surface of the tubing. Apart from heaters, heat-exchange appliances that circulate a heating fluid may be utilized as the heat source 9. In Embodiment 4, the plurality of recirculation-path 6a systems may each be furnished with a dedicated pump 7.

Only selected embodiments have been chosen to illustrate the present invention. To those skilled in the art, however, it will be apparent from the foregoing disclosure that various changes and modifications can be made herein without departing from the scope of the invention as defined in the appended claims. Furthermore, the foregoing description of the embodiments according to the present invention is provided for illustration only, and not for limiting the invention as defined by the appended claims and their equivalents.

1. A method for continuous amplification of DNA, the method repeating DNA amplification reactions continuously by:
with a recirculation path and a pump furnished in the recirculation path, feeding unidirectionally through the recirculation path a reaction mixture containing DNA fragments and a reagent solution, held in a reaction-mixture tank;
sending the reaction mixture within the recirculation path by way of, in order, a denaturing isothermal tank wherein a temperature for dissolving apart the DNA's double strands is maintained, an annealing isothermal tank wherein a temperature at which primers contained in the reagent solution anneal to the DNA fragments is maintained, and an elongation isothermal tank wherein a temperature at which complementary chains are extended continuously onto the primers is maintained; and
with heat-exchange fluids within the isothermal tanks, maintaining the reaction mixture for predetermined times at predetermined temperatures, and subsequently recirculating the reaction mixture into the reaction-mixture tank.

2. The DNA continuous amplification method set forth in claim 1, wherein the amplification reactions are performed by making time for heat-exchange in the denaturing isothermal tank a reference time, and setting individual heat-exchange times in the annealing isothermal tank and the elongation isothermal tank as multiples of the reference time.

3. A method for continuous amplification of DNA, the method repeating DNA amplification reactions continuously by:
circuit-feeding unidirectionally through an endless recirculation path, using a pump provided therein, a reaction mixture containing DNA fragments and a reagent solution, held within the recirculation path;
circulating the reaction mixture within the recirculation path by way of, in order, a denaturing isothermal tank wherein a temperature for dissolving apart the DNA's double strands is maintained, an annealing isothermal tank wherein a temperature at which primers contained in the reagent solution anneal to the DNA fragments is maintained, and an elongation isothermal tank wherein a temperature at which complementary chains are extended continuously onto the primers is maintained; and
with heat-exchange fluids within the isothermal tanks, maintaining the reaction mixture for predetermined times at predetermined temperatures.

4. An apparatus for continuous amplification of DNA, comprising:
a reaction-mixture tank for holding a reaction mixture containing DNA fragments and a reagent solution;
a denaturing isothermal tank for holding a heat-exchange fluid adjusted to a temperature for dissolving apart the DNA's double strands;
an annealing isothermal tank for holding a heat-exchange fluid adjusted to a temperature at which primers contained in the reagent solution anneal to the DNA fragments;
an elongation isothermal tank for holding a heat-exchange fluid adjusted to a temperature at which complementary chains are extended continuously onto the primers;
a recirculation-path system through which the reaction mixture in the reaction-mixture tank is fed and guided, the recirculation-path system being arranged such that it circuits from the reaction-mixture tank and goes by way of the denaturing isothermal tank, the annealing isothermal tank, and the elongation isothermal tank back to the reaction-mixture tank; and
a pump working to feed the reaction mixture in said recirculation-path system unidirectionally through it; wherein
the apparatus is configured such that the reaction mixture in said recirculation-path system is for timed intervals maintained at prescribed temperatures determined by the heat-exchange fluids in the isothermal tanks.

5. The DNA continuous amplification apparatus set forth in claim 4, wherein said denaturing isothermal tank, said annealing isothermal tank, and said elongation isothermal tank each respectively include:
a container body for holding the heat-exchange fluid;
a heat source for heating the heat-exchange fluid to, and retaining it at, the prescribed temperatures; and
a stirring device for stirring the heat-exchange fluid.

6. The DNA continuous amplification apparatus set forth in claim 4, wherein said denaturing isothermal tank, said annealing isothermal tank, and said elongation isothermal tank each respectively include:
a container body for holding the heat-exchange fluid;
a stirring device for stirring the heat-exchange fluid; and
a heating device containing a pump for circuit-feeding the heat-exchange fluid in between the container body and the heating device, and a heat source for heating the heat-exchange fluid to and retaining it at prescribed temperatures, wherein said heating device supplies the heat-exchange fluid to said container body.

7. The DNA continuous amplification apparatus set forth in claim 4, wherein a plurality of said recirculation-path systems in parallel is provided along with said pump between the reaction-mixture tank and said container bodies.

8. The DNA continuous amplification apparatus set forth in claim 5, wherein a plurality of said recirculation-path systems in parallel is provided along with said pump between the reaction-mixture tank and said container bodies.

9. The DNA continuous amplification apparatus set forth in claim 6, wherein a plurality of said recirculation-path systems in parallel is provided along with said pump between the reaction-mixture tank and said container bodies.

10. The DNA continuous amplification apparatus set forth in claim 4, further comprising:
a plurality of sets of said denaturing isothermal tank, said annealing isothermal tank, and said elongation isothermal tank; and
a plurality of said recirculation-path systems in parallel, provided along with said pump between the reaction-mixture tanks and the isothermal tanks of said plurality of sets.

11. The DNA continuous amplification apparatus set forth in claim 5, further comprising:
a plurality of sets of said denaturing isothermal tank, said annealing isothermal tank, and said elongation isothermal tank; and
a plurality of said recirculation-path systems in parallel, provided along with said pump between the reaction-mixture tanks and the isothermal tanks of said plurality of sets.

12. The DNA continuous amplification apparatus set forth in claim 6, further comprising:
a plurality of sets of said denaturing isothermal tank, said annealing isothermal tank, and said elongation isothermal tank; and
a plurality of said recirculation-path systems in parallel, provided along with said pump between the reaction-mixture tanks and the isothermal tanks of said plurality of sets.

13. The DNA continuous amplification apparatus set forth in claim 4, further comprising a coiled heat-exchange path immersed into each of said isothermal tanks in sections along the way of said recirculation-path system.

14. The DNA continuous amplification apparatus set forth in claim 5, further comprising a coiled heat-exchange path immersed into each of said isothermal tanks in sections along the way of said recirculation-path system.

15. The DNA continuous amplification apparatus set forth in claim 6, further comprising a coiled heat-exchange path immersed into each of said isothermal tanks in sections along the way of said recirculation-path system.

16. The DNA continuous amplification apparatus set forth in claim 7, further comprising a coiled heat-exchange path immersed into each of said isothermal tanks in sections along the way of said recirculation-path system.

17. The DNA continuous amplification apparatus set forth in claim 10, further comprising a coiled heat-exchange path immersed into each of said isothermal tanks in sections along the way of said recirculation-path system.
